(19) United States
(12) Patent Application Publication
(10) Pub. No.: US 2026/0028573 A1
(43) Pub. Date: Jan. 29, 2026

(54) ASSEMBLING AND GROWING CELLULAR OBJECTS ON A CONTACT STRUCTURE BY MEANS OF AXIAL AND TRANSVERSE ACOUSTIC RADIATION FORCES

(71) Applicants: CENTRE NATIONAL DE LA RECHERCHE SCIENTIFIQUE, PARIS (FR); ECOLE SUPERIEURE DE PHYSIQUE ET DE CHIMIE INDUSTRIELLES DE LA VILLE DE PARIS, PARIS (FR); UNIVERSITE PARIS CITE, Paris (FR); INSTITUT NATIONAL DE LA SANTÉ ET DE LA RECHERCHE MEDICALE, Paris (FR); SORBONNE UNIVERSITE, PARIS (FR)

(72) Inventors: Jean-Luc AIDER, Vanves (FR); Nathan JEGER-MADIOT, PARIS (FR); Jean-Michel PEYRIN, Paris (FR); Mauricio HOYOS, Créteil (FR); Chloé DUPUIS, Paris (FR)

(73) Assignees: CENTRE NATIONAL DE LA RECHERCHE SCIENTIFIQUE, PARIS (FR); ECOLE SUPERIEURE DE PHYSIQUE ET DE CHIMIE INDUSTRIELLES DE LA VILLE DE PARIS, PARIS (FR); UNIVERSITE PARIS CITE, Paris (FR); INSTITUT NATIONAL DE LA SANTÉ ET DE LA RECHERCHE MEDICALE, Paris (FR); SORBONNE UNIVERSITE, PARIS (FR)

(21) Appl. No.: 18/995,506

(22) PCT Filed: Jul. 6, 2023
(86) PCT No.: PCT/EP2023/068715

§ 371 Date: Jan. 16, 2025

(30) Foreign Application Priority Data
Jul. 21, 2022 (FR) FR2207467

(51) Int. Cl. C12M 1/42; C12M 1/00; C12M 1/12

(52) U.S. Cl. CPC C12M 35/04; C12M 23/34; C12M 25/02; C12M 25/14

(57) ABSTRACT

A device and method for assembling objects by generating an axial acoustic radiation force in order to position the objects on pressure nodes and/or antinodes and generating a transverse acoustic radiation force in order to move the thus positioned objects on a porous membrane contact structure.

### TECHNICAL FIELD

The invention relates to the field of biotechnology and in particular assembling cells or micrometric particles, for example with a view to reconstructing or modeling living tissue.

The invention is of particular interest in, but is by no means limited to, the fields of cell therapy, pharmacological modeling, food processing, for example for meat, micro-algae or plant cultivation, or even aerospace, notably for cell culture in microgravity conditions.

### PRIOR ART

In the context of research into organ-on-a-chip and organoid reconstruction and modeling, an increasing number of experimental approaches are aimed at structuring cell assemblies.

The techniques most widely used for this purpose include cell manipulation within microfluidic devices and tissue formation by additive manufacturing.

Another known technique, described in the following document, consists of structuring cell sheets by acoustic levitation in hydrogels: Bouyer et al. A bio-Acoustic Levitational (BAL) Assembly Method for Engineering of Multilayered, 3D Brain-Like Constructs, Using Human Embryonic Stem Cell Derived Neuro-Progenitors, Adv. Mater. 2016, 28, 161-167. This technique allows cells to be assembled in a hydrogel in the form of layers or sheets with the aim of establishing connections between cells in different layers. However, this technique does not allow the development of such connections to be controlled in a satisfactory manner.

More generally, known assembly techniques in this field are complex and expensive, can require long times to assemble and culture cells and, to the extent that they are implemented in vitro, can result in the death of a large number of cells.

### DISCLOSURE OF THE INVENTION

The present invention aims to overcome the aforementioned disadvantages by providing a device for assembling objects, comprising:
a cavity configured to receive a fluid and the objects,
a generating system configured to generate in the cavity, along an axial direction, a stationary acoustic wave so as to produce an axial acoustic radiation force suitable for positioning objects on pressure nodes and/or antinodes formed by the acoustic wave.

According to the invention, the device comprises a contact structure forming one or more surfaces extending along the axial direction and the generating system is configured to exert a transverse acoustic radiation force capable of displacing the objects towards the surface(s) of the contact structure.

The propagation of a standing acoustic wave in the cavity allows one or more pressure nodes, that is, places where the pressure of the fluid is zero, and one or more antinodes or pressure antinodes, that is, places where this pressure is maximum, to be formed in the cavity along the axial direction.

In a manner known per se, based on the properties of the objects and in particular their density-compressibility factor, or acoustic contrast with respect to the fluid contained in the cavity, the axial acoustic radiation force moves the objects either towards a pressure node when their acoustic contrast is positive or towards a pressure antinode when it is negative.

The axial acoustic radiation force thus makes it possible to form one or more aggregates of objects in the cavity, one after the other along the axial direction, extremely quickly—typically in a few seconds—and using particularly simple and inexpensive equipment.

The transverse acoustic radiation force, which is preferably generated after such axial positioning of the objects, brings each of the aggregates thus formed into contact with the contact structure.

The invention thus makes it possible to produce one or more assemblies of objects and to maintain these assemblies in acoustic levitation against the contact structure.

By maintaining such assemblies in acoustic levitation for the required duration, for example several hours or days, it is possible to promote interactions between objects when said objects are living, in particular when said objects are biological cells.

The invention thus makes it possible to produce a cell culture in acoustic levitation, by controlling, for each of the assemblies, the development of connections and interactions between the cells of this assembly and/or between these cells and other objects or elements that may be arranged on or in the contact structure (see below).

In the present description, an “object” refers to a living or inert element preferably of small size with respect to the length of the standing acoustic wave generated in the cavity.

By way of a non-limiting example, the objects may have a micrometric size, for example between 1 μm and 100 μm, typically when the frequency of the standing wave has a value in the MHz range.

For another example, notably when the generating frequency is in the KHz range, the objects can be millimetric in size, for example between 1 mm and 100 mm.

However, the invention can also be applied to objects of other sizes. For example, some or all of the objects may be less than 1 μm in size, by being for example formed by bacteria or viruses, and/or be several hundred μm in size. Furthermore, some or all of the objects may be multi-cellular elements or artificially formed objects or even objects taken from an organ.

The fluid wherein the objects are suspended is preferably a liquid which, depending on the application envisaged, may comprise water or form a culture medium or more generally an aqueous medium comprising, for example, a hydrogel prepolymer or colloidal particles.

The invention also provides a solution that is particularly precise in terms of positioning objects in space and, if necessary, allows the development of intercellular interactions to be controlled.

The device of the invention can thus form an acoustofluidic chip that can be used for a variety of applications and have varied structural characteristics, in particular with regard to its contact structure.

In one embodiment, the contact structure comprises one or more membranes or walls.

In the present description, a membrane, also referred to as a “wall”, is a structure typically comprising two relatively large surfaces with respect to a thickness of this structure, that is, with respect to the distance separating these two surfaces.

In one embodiment, the cavity comprises several chambers delimited by one or more of said membranes.

By way of a non-limiting example, the cavity may comprise two chambers and the contact structure may comprise a single membrane arranged so that a first surface of the membrane delimits one of these chambers and a second surface of the membrane delimits the other chamber.

In one embodiment, each of the chambers comprises a respective part of the objects.

Thus, in the aforementioned example of compartmentalizing the cavity into two chambers, the objects can be divided into two series, with objects of the first series being placed in one of the chambers and objects of the second series in the other chamber.

In this non-limiting example, the transverse acoustic radiation force can be configured to move the objects of the first series towards the membrane in order to group these objects against the first surface of the membrane and to move the objects of the second series towards the membrane in order to group these objects against the second surface of the membrane.

The objects of the first series and of the second series, arranged at a same position along the axial direction, can thus be grouped together on both sides of the membrane, allowing, for example, the development of interactions, particularly through the membrane when said membrane is porous.

Thus, in one embodiment, one or more of said membranes are porous.

In one embodiment, one or more of said surfaces formed by the contact structure are surfaces each extending around a respective direction.

Preferably, this direction is parallel or oblique with respect to said axial direction.

For example, one or more of said surfaces formed by the contact structure may each extend around a respective direction so as to form a surface of revolution around this direction, for example so as to present a cylindrical geometry.

Such a surface of revolution may have a relatively small cross-section with respect to the length of the standing acoustic wave generated in the cavity and/or with respect to the axial dimension of this surface of revolution, so as to allow one or more assemblies of objects around such a surface, for example in the form of spheroid or ovoid assemblies.

In one embodiment, one or more of said surfaces formed by the contact structure can each form a hollow structure, allowing, for example, the infusion and/or removal of biological or chemical objects, elements or samples, or even to encapsulate objects such as biological cells in the contact structure.

Thus, in one embodiment, the device comprises elements, such as biological cells, connected to the contact structure so as to allow interactions between these elements and one or more of said objects when these latter are moved towards the surfaces of the contact structure.

Of course, these principles can be generalized so that one or more of said surfaces of the contact structure, whether said contact structure takes the form of one or more membranes and/or hollow structures and/or arranged around one or more directions and/or one or more three-dimensional structures of any geometry, may be cellularized or, more generally, comprise or carry elements containing biological, physico-chemical and/or physical information, for example using molecules that promote or inhibit cell multiplication, nanoparticles or even micrometric or nanometric texturing elements.

In one embodiment, the contact structure comprises or forms one or more electrodes and/or has conductive properties.

In one embodiment, the contact structure comprises a gas-permeable material, for example polydimethylsiloxane, for example so as to diffuse oxygen into the core of the object assembly or assemblies.

In one embodiment, the contact structure comprises one or more wires forming one or more of said surfaces.

In one embodiment, the generating system comprises one or more piezoelectric and/or ultrasonic transducers which may comprise a plurality of matrix-organized elements and/or one or more acoustic holographic lenses.

In one embodiment, said objects comprise objects having a positive acoustic contrast with respect to the fluid so that the axial acoustic radiation force moves these objects towards the pressure nodes and/or objects having a negative acoustic contrast with respect to the fluid so that the axial acoustic radiation force moves these objects towards the pressure antinodes.

The invention also relates a method for assembling objects using a device as defined hereinbefore.

This method comprises generating the axial acoustic radiation force so as to position the objects on the pressure nodes and/or antinodes and generating the transverse acoustic radiation force so as to move the objects towards the surface(s) of the contact structure.

The transverse acoustic radiation force is preferably generated after objects are positioned on the pressure nodes and/or antinodes, for example with a generating system comprising a matrix of piezoelectric elements.

Alternatively, the axial and transverse acoustic radiation forces can be generated simultaneously, for example with a generating system comprising a single transducer or an acoustic holographic lens.

The method may implement any number of steps allowing different combinations of the functional characteristics described hereinbefore to be obtained, based notably on the structural characteristics of the device and notably the contact structure.

Without limitation, the invention does not cover, at least in some embodiments, applications wherein said objects comprise human embryonic stem cells involving the destruction of a human embryo and applications wherein the invention would be implemented so as to constitute or develop a human body.

Further advantages and features of the invention will become apparent from the following detailed, non-limiting description.

### BRIEF DESCRIPTION OF THE DRAWINGS

The following detailed description makes reference to the accompanying drawings in which:

FIG. 1 is a schematic view, in axial cross-section, of a device according to the invention, this device comprising a cavity, a porous membrane arranged in the cavity so as to delimit two chambers, an ultrasonic transducer comprising a matrix of piezoelectric elements and a reflector, each of the chambers containing cellular objects held in acoustic levitation in the form of aggregates at a distance from the membrane;

FIG. 2 is a schematic view of the device of FIG. 1, with the aggregates of cellular objects being arranged against the membrane to form aggregate assemblies capable of developing intercellular interactions therebetween through membrane pores;

FIG. 3 is a schematic cross-sectional view of a device according to the invention which differs from that of FIG. 1 in that it comprises two porous membranes arranged in the cavity so as to delimit four chambers, each of which contains cellular objects held in acoustic levitation in the form of aggregates at a distance from the membrane;

FIG. 4 is a schematic view of the device of FIG. 4, with the aggregates of cellular objects being arranged against the membranes to form aggregate assemblies capable of developing intercellular interactions therebetween through membrane pores;

FIG. 5 is a schematic view, in axial cross-section, of a device according to the invention, this device comprising a cavity, elongated contact elements arranged in the cavity, an ultrasonic transducer comprising a matrix of piezoelectric elements and a reflector, the cavity containing cellular objects held in acoustic levitation in the form of aggregates at a distance from the contact elements;

FIG. 6 is a schematic view of the device of FIG. 5, with the aggregates of cellular objects being arranged around the contact elements.

### DETAILED DESCRIPTION OF EMBODIMENTS

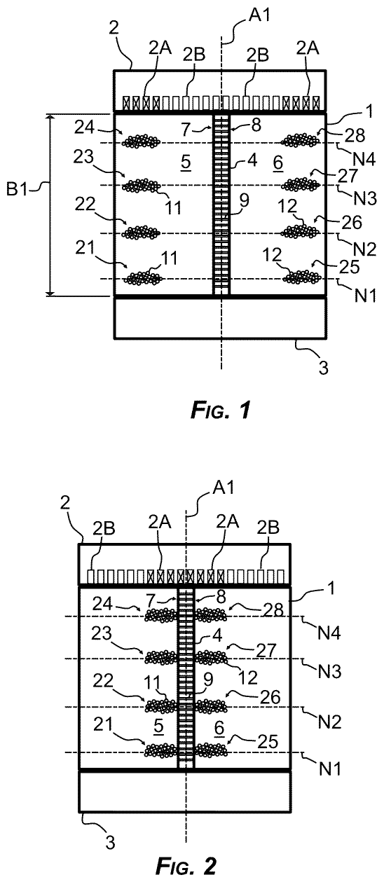

FIG. 1 schematically shows an example of a device according to a first embodiment of the invention.

This device comprises a container which forms a cavity 1 suitable for containing a fluid and/or various substances, particularly in liquid form.

Typically, the cavity 1 extends along an axial direction A1, which corresponds to a vertical direction in this example. Along direction A1, the cavity 1 has a dimension B1 defining a height of the cavity 1.

In this example, the cavity 1 has a generally cylindrical shape, with direction A1 forming an axis of symmetry of the cavity 1. In variants not shown, the cavity 1 can have another geometry, for example a rectangular cross-section.

The device of the invention also comprises a system for generating acoustic waves, namely a transducer 2 equipped with a matrix of piezoelectric elements 2A and 2B, and an acoustic reflector 3.

Referring to FIG. 1, the transducer 2 is arranged at a first end of the cavity 1 along direction A1, in this case vertically above the cavity 1. The reflector 3 delimits a second end of the cavity 1 along direction A1, it being arranged in this case vertically below the cavity 1.

The piezoelectric elements 2A and 2B are distributed transversely, that is, across the width of the cavity 1, so that each of these elements is located at a respective position radially with respect to direction A1.

This system is configured to generate a standing acoustic wave in the cavity 1 and propagate said wave in the fluid contained therein, along an axial propagation direction corresponding to direction A1.

The standing wave can be generated by one or more of the piezoelectric elements of the transducer 2, with a frequency identical to the resonant frequency of the cavity 1, which in this case forms a resonator. Alternatively, the standing wave can have a frequency different from the resonant frequency of the cavity 1.

In all cases, the system is configured to be able to generate, notably, a wave having a wavelength A less than or equal to twice the height B1 of the cavity 1, in order to form along direction A1 at least one pressure node and at least one pressure antinode.

The transducer 2, in this example, is a broadband transducer equipped with an ultrasonic source. Such a transducer 2 can be used to modify the position of the standing wave nodes along direction A1 and/or the distance between these nodes, by adjusting the frequency of the wave.

In the embodiment of FIG. 1, the device comprises a wall 4, also known as a “membrane”, which extends into cavity 1 so as to separate the cavity 1 into two chambers 5 and 6.

The membrane 4 comprises a first surface 7 which delimits the chamber 5 and a second surface 8 which delimits the chamber 6.

The membrane 4 and the surfaces 7 and 8 thereof extend along direction A1, in this case parallel to this direction.

In this example, the membrane 4 is made of nitrocellulose and comprises pores 9 forming openings which pass right through the membrane 4 so as to open onto surfaces 7 and 8.

By way of illustration, the membrane 4 may have a thickness, defined as the distance between surfaces 7 and 8, of 150 μm and pores having a diameter of 3 μm. Without limitation, the size of such pores can range from the nanometric scale to the micrometric scale.

The chambers 5 and 6 are in this example filled with a fluid containing objects 11 and 12 each having a size between 1 μm and 100 μm.

In this non-limiting example, objects 11 are biological cells of a first type which are received in chamber 5 while objects 12 are biological cells of a second type received in chamber 6. The fluid forms a culture medium for cell objects 11 and 12.

Without limitation, objects 11 and 12 can be neurons or glial, tumor, endothelial, epithelial, bone or even immune cells.

In this specific example, each of the objects 11 and 12 has a density Po greater than the density pr of the fluid. The objects 11 and 12 are further selected so that the speed co of propagation of an acoustic wave in these objects 11 and 12 is greater than the speed cr of propagation of this acoustic wave in the fluid.

In this example, the device of FIG. 1 is implemented in such a way that objects 11 and 12 can be structured as described below.

In an initial state, not shown, objects 11 and 12 are suspended in the fluid, respectively in chambers 5 and 6.

The transducer 2 is implemented so as to generate a standing acoustic wave in cavity 1, along direction A1, in this example with a wavelength forming four pressure nodes N1-N4. In this case, the wave is generated via piezoelectric elements 2A, identified by rectangles with a cross on FIG. 1, with piezoelectric elements 2B, identified by rectangles without a cross on FIG. 1, being inactivated.

This standing wave produces an axial acoustic radiation force that acts on objects 11 and 12.

This axial acoustic radiation force FRA_{A} can notably be described according to the following model, known per se, by K. Yosioka and Y. Kawasima:

$${FRA}_{A} = \frac{π}{8} {ρ}_{f} {v}_{0}^{2} {kd}^{3} {F}_{y} sin ( k z )$$

where v₀ is the wave velocity, k the wave number, F_{y} the acoustic contrast factor, or density-compressibility, and z the axial position of object 11 or 12 considered along direction A1, that is, along the direction of wave propagation.

The acoustic contrast factor, or density-compressibility, F_{y} can be defined as follows:

$${F}_{y} = \frac{1 + \frac{2}{3} ( 1 - \frac{{ρ}_{f}}{{ρ}_{o}} )}{2 + \frac{{ρ}_{f}}{{ρ}_{o}}} - \frac{{ρ}_{f} {c}_{f}^{2}}{3 {ρ}_{o} {c}_{o}^{2}}$$

where ρₒ is the density of object 11 or 12 in question and co is the propagation speed of the wave within object 11 or 12 in question.

Given the density and propagation speed of the acoustic wave of objects 11 and 12 with respect to the fluid, objects 11 and 12 have a positive density-compressibility, or acoustic contrast factor.

Given the aforementioned respective properties of the fluid and objects 11 and 12, from said initial state wherein objects 11 and 12 are distributed relatively evenly throughout cavity 1, the axial acoustic radiation force causes objects 11 and 12 to move towards the nodes of the standing acoustic wave, so as to reach a configuration as shown in FIG. 1.

The axial acoustic radiation force thus results in a positioning of objects 11 and 12 in the form of groups, also known as “aggregates”, as shown in FIG. 1, which are located radially at the activated piezoelectric elements 2A.

Thus, a group 21 of objects 11 is formed axially at pressure node N1 and radially at the piezoelectric elements 2A facing the chamber 5. Three other groups 22, 23 and 24 of objects 11 form axially at pressure nodes N2, N3 and N4, respectively, and radially at the same level as group 21.

Symmetrically, a group 25 of objects 12 is formed axially at pressure node N1 and radially at the piezoelectric elements 2A facing the chamber 6. Three other groups 26, 27 and 28 of objects 12 form axially at pressure nodes N2, N3 and N4, respectively, and radially at the same level as group 25.

The activated piezoelectric elements 2A can maintain objects 11 and 12 in acoustic levitation for the time required to achieve sufficient self-organization of aggregates 21 to 28, which in this example constitute substantially ovoid three-dimensional structures.

The transducer 2 can then be controlled to produce a transverse acoustic radiation force in order to move objects 11 and 12 towards the membrane 4, in particular so that aggregates 21 to 24, located in chamber 5, are pressed against the surface 7 of the membrane 4 and aggregates 25 to 28, located in chamber 6, are pressed against the surface 8 of membrane 4.

In this example, such a transverse acoustic radiation force is achieved by changing the activation state of the piezoelectric elements 2A and 2B, that is, by progressively activating the piezoelectric elements radially in the direction of direction A1 and by inactivating the previously activated piezoelectric elements as new piezoelectric elements are activated.

The transducer 2 thus allows the acoustic field in cavity 1 to be spatially controlled so as to exert a transverse acoustic radiation force capable of translating aggregates 21 to 28 towards membrane 4, until a configuration such as that shown in FIG. 2 is reached.

In a manner known per se, the transverse acoustic radiation force FRA_{T} can be expressed as a function of the gradient of the acoustic energy density:

$$F R {A}_{T} ( x , y , z ) = {d}^{3} \frac{3 ( {ρ}_{o} - {ρ}_{f} )}{{ρ}_{f} + 2 {ρ}_{o}} ∇ 〈 {E}_{a c} ( x , y , z ) 〉$$

where d is the diameter of objects 11 and 12 and ∇ [symbol] E_{ac}(x,y, z [symbol] > is the acoustic energy density gradient.

In the configuration of FIG. 2, the piezoelectric elements 2A located radially at the membrane 4 can remain activated so as to keep the aggregates 21 to 28 in contact with the membrane 4, by acoustic levitation.

The invention thus makes it possible to assemble object structures, in this example aggregates 21-24 of objects 11 of a first type with aggregates 25-28 of objects 12 of a second type, without the different types of objects located in the same acoustic levitation plane coming into direct contact with each other and so that contact can in this example be established via the porous membrane 4.

Such a membrane 4 notably allows interactions between objects 11 and 12 to be controlled through pores 9 which can provide passages for axon-type connections when objects 11 and 12 are primary neurons.

Numerous variations can be made to the device just described and to the implementation thereof. For example, the height B1 of cavity 1 and/or the frequency of the standing acoustic wave can be modified in order to increase the number of pressure nodes, for example so as to assemble several dozen aggregates simultaneously.

The size and shape of objects 11 and 12 can also be modified, as can the concentration of objects in cavity 1, the volume of cavity 1 or even the frequency of the wave, in order, for example, to determine a number of objects per aggregate or assembly and the size of the resulting assembly or assemblies. The choice of amplitude and of frequency of the acoustic wave, setting the magnitude of the acoustic radiation force, can also be controlled to modify the lateral and/or axial dimensions of the assembly or assemblies. The amplitude of the acoustic pressure applied to the assemblies of objects can also be controlled, for example to force a given spatial organization or even to stimulate the objects and thus force certain spatial organizations and/or functionalities.

As another example, the membrane 4 may be a glass wall or more generally a non-porous structure, notably allowing different fluids to be used in each of chambers 5 and 6 of the cavity 1. Such a wall can, of course, be functionalized and/or cellularized and/or be gas-permeable.

Other non-limiting embodiments of the invention are described below, it being understood that the foregoing description applies by analogy to these various embodiments and to variants thereof. In the following description, these embodiments are therefore described essentially in terms of their differences with respect to the embodiment shown in FIGS. 1 and 2.

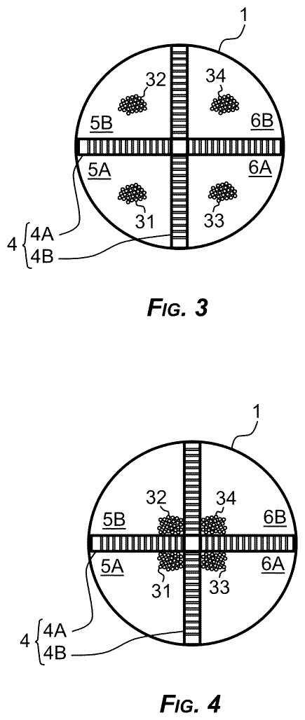

In the embodiment shown in FIGS. 3 and 4, the contact structure 4 comprises not one membrane but two porous membranes 4A and 4B separating cavity 1 into four chambers 5A, 5B, 6A and 6B. Each of chambers 5A, 5B, 6A and 6B comprises objects 31, 32, 33 and 34, respectively, of a different nature.

Such a device makes it possible to form complex assemblies, in this case doubling the types of objects with respect to the device shown in FIG. 1.

More generally, the contact structure 4 can therefore be configured to compartmentalize the acoustic levitation cavity into several independent chambers, with different culture media, as required. In particular, this allows one cell type per chamber to be maintained in acoustic levitation and cultured in three dimensions, all in a single acoustofluidic chip.

By controlling the frequency of the acoustic waves emitted and/or the shape of the reflector, and consequently the spatial pattern of the acoustic field generated in the cavity, levitating cell objects can be brought into contact with a contact structure such as a membrane, either at the start of culture or during culture.

In these various examples, different symmetrical or asymmetrical shapes of reflector 3 can be implemented to promote a given spatial organization of the aggregates of acoustically levitated objects, for example an organization in sheets, spheroid or ovoid structures, rings or even independent lobes.

The membrane(s) of the contact structure can be of various physico-chemical types, for example be formed of a hydrogel, an elastomer or even an inorganic material, and/or can comprise micrometric or nanometric size textures.

The use of membranes with controlled porosity makes it possible to control on the one hand the self-assembly of different aggregates, notably spheroids, and thus to reconstruct complex multicellular assemblies, and on the other hand the nature of interactions between aggregates of various types. For example, it is possible to control solute exchanges, notably with membrane pores of submicrometer size, cell extensions including neuron axons, notably with a porosity of between 1 μm and 5 μm, or even cell or object exchanges, notably with a porosity greater than 5 μm.

Furthermore, the chamber(s) delimited by one or more membranes, or more generally by a contact structure that can be otherwise constituted, can be used solely for initial structuring steps, that is, object assemblies, these assemblies can then be cultured in a conventional manner, for example in a liquid medium or in a hydrogel. Alternatively, the device of the invention can be used to perform long-term culture in acoustic levitation.

In the embodiment shown in FIGS. 1 and 2, the transverse acoustic radiation force is generated by selective activation of piezoelectric elements in the transducer 2. This force can of course be generated using another type of acoustic field control member, for example with an acoustic holographic lens. As is well known, an acoustic holographic lens can be coded at several frequencies. Thus, a first frequency can be used to organize the objects into aggregates as shown in FIG. 1 and a second frequency to bring these aggregates back and hold them against the contact structure 4 to form assemblies as shown in FIG. 2. More generally, the wave generating system producing the axial and transverse acoustic radiation forces may comprise one or more transducers or combinations of transducers of different types and/or positioned axially and/or transversely.

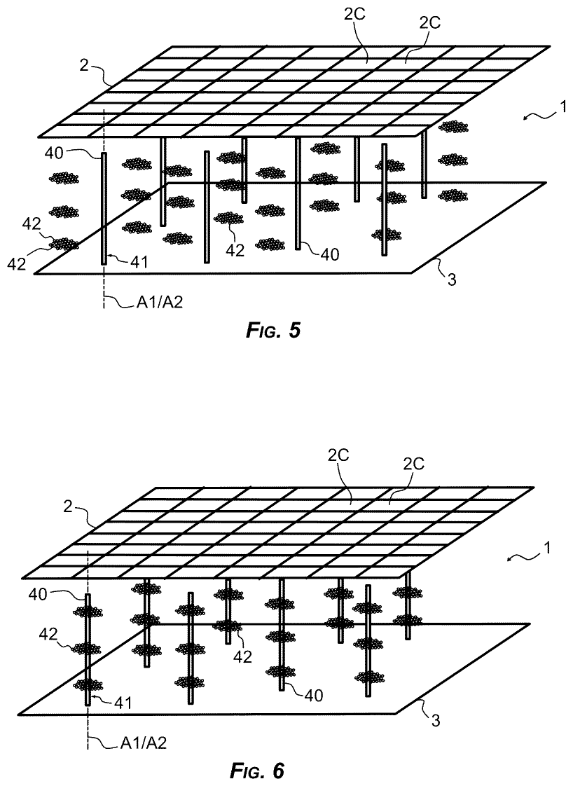

Another type of contact structure is shown in FIGS. 5 and 6.

Referring to FIG. 5, the device comprises a contact structure comprising an array of contact elements 40 each of which has an elongated geometry along axial direction A1 of the cavity 1.

In this non-limiting example, each of the contact elements 40 has a cylindrical geometry so as to form an external surface 41 extending around a direction A2 parallel to the axial direction A1 of cavity 1.

Similar to the embodiment shown in FIGS. 1 and 2, the acoustic wave generating system of the device shown in FIG. 5 also comprises an ultrasonic transducer 2 provided with piezoelectric elements 2C organized in a matrix and a reflector 3 each extending in a plane transverse to cavity 1 so as to be able to move objects 42 present in the cavity 1.

From an initial state, not shown, wherein the objects 42 are suspended in the fluid, the axial acoustic radiation force resulting from the standing wave generated by the transducer 2 causes the objects 42 to be positioned in the form of aggregates as shown in FIG. 5.

Aggregates of objects 42 are formed in this particular example on three pressure nodes formed by the standing wave, the objects 42 presenting a positive acoustic contrast with respect to the fluid, transversely facing activated piezoelectric elements 2C, in this configuration at a distance from the contact elements 40.

Similar to the embodiment shown in FIGS. 1 and 2, the activation of the piezoelectric elements 2C is then modified to produce a transverse acoustic radiation force causing a movement of aggregates around contact elements 40 as shown in FIG. 6, so as to form in this example spheroid assemblies that can thus be cultured.

The contact elements 40 can be of different physico-chemical and geometric types. For example, they can be solid or hollow, permeable or non-permeable, electroactive or non-electroactive, making it possible to perfuse, stimulate, monitor and/or sample assemblies in a controlled manner.

Thus, in one alternative embodiment, the contact elements 40 can form electrodes intended to measure or impose electrical activity. Electro-active and/or conductive contact elements 40 can notably be used to model a vascularization phenomenon, with electrical stimulation and recording of the electrical activity of the assemblies of objects 42.

In one alternative embodiment, the contact elements 40 can be hollow and/or porous, for example in order to allow solutes such as biological and physical compounds or agents, cells or even viruses to be injected and trapped in the contact elements 40 and perfused into the assemblies of objects 42. The nature of the diffusion/migration of such solutes can be controlled through the porosity of the contact elements 40.

Such alternatives can be implemented or combined with other types of contact structures, for example one or more membranes 4 as described hereinbefore with reference to FIGS. 1 to 4.

More generally, the contact structure of the device of the invention can be functionalized so as to modify the interaction between cellular objects assembled on the contact structure.

In addition, functionalization of the contact structure may comprise grafting objects onto one or more surfaces of the contact structure and/or inside the contact structure, for example inside contact elements 40 as shown in FIGS. 5 and 6. The grafted objects may be particles conferring new properties on the contact structure, for example in terms of electrical, magnetic or even acoustic conductivity, bioactive substances influencing the development of cellular objects, or even cells, for example endothelial or astrocytic, or other living organisms.

In an alternative embodiment, the contact elements 40 of the device shown in FIGS. 5 and 6 can be formed by a cured hydrogel, for example by confocal photopolymerization. These contact elements 40 may comprise cellular or functionalization objects, such as those described hereinbefore, trapped in the hydrogel forming these contact elements 40. Of course, such contact elements can have any more or less complex three-dimensional geometry, forming, for example, a lattice structure or the like.

The invention allows a wide range of applications, including the reconstruction of complex tissue elements, the modeling of multi-organ interactions in series, for example interactions between different areas of the brain, and the series or parallel arrangement of organ models. Among other applications, the invention allows three-dimensional reconstruction of neuroanatomical pathways or three-dimensional models of tissue barriers of the blood-brain or placental type. In the context of neuroscience, the anisotropy of the porosity, for example conical holes, of membranes could be used to control the direction of growth of axons and/or nerves from one organoid to another. As another example, complex biological interfaces can be modeled by cellularizing membranes, porous or otherwise, notably by pre-seeding membranes with cells of interest.

1-10. (canceled)

11. A device for assembling objects, comprising:
a cavity configured to receive a fluid and the objects,
a generating system configured to generate in the cavity, along an axial direction, a standing acoustic wave so as to produce an axial acoustic radiation force suitable for positioning objects on pressure nodes and/or antinodes formed by the acoustic wave,
characterized in that it comprises a contact structure forming one or more surfaces extending along the axial direction and in that the generating system is configured to exert a transverse acoustic radiation force capable of displacing the objects towards the surface(s) of the contact structure.

12. The device according to claim 11, wherein the contact structure comprises one or more membranes.

13. The device according to claim 12, wherein the cavity comprises several chambers delimited by one or more of said membranes, each of the chambers preferably comprising a respective part of the objects.

14. The device according to claim 12, wherein one or more of said membranes are porous.

15. The device according to claim 11, wherein one or more of said surfaces formed by the contact structure are surfaces each extending around a respective direction, this direction preferably being parallel or oblique with respect to said axial direction.

16. The device according to claim 11, wherein the contact structure comprises or forms one or more electrodes.

17. The device according to claim 11, comprising elements, such as biological cells, connected to the contact structure in such a way as to allow interactions between these elements and one or more of said objects when said objects are moved towards the surfaces of the contact structure.

18. The device according to claim 11, wherein the generating system comprises one or more piezoelectric and/or ultrasonic transducers which can comprise several matrix-organized elements and/or one or more acoustic holographic lenses.

19. The device according to claim 11, wherein said objects comprise objects having a positive acoustic contrast with respect to the fluid so that the axial acoustic radiation force moves these objects towards the pressure nodes and/or objects having a negative acoustic contrast with respect to the fluid so that the axial acoustic radiation force moves these objects towards the pressure antinodes.

20. A method for assembling objects using a device according to claim 11, comprising generating the axial acoustic radiation force so as to position the objects on the pressure nodes and/or antinodes and generating the transverse acoustic radiation force so as to move the objects towards the surface(s) of the contact structure.
